(19) United States
(12) Patent Application Publication
(10) Pub. No.: US 2026/0260417 A1
(43) Pub. Date: Sep. 3, 2026

(54) SYSTEMS, METHODS, AND APPARATUSES FOR LEARNING ANATOMICAL CONSISTENCY, SUB-VOLUME SPATIAL RELATIONSHIPS AND FINE-GRAINED APPEARANCE FOR COMPUTED TOMOGRAPHY IMAGES

(71) Applicants: Jiaxuan PANG, Tempe, AZ (US); DongAo MA, Tempe, AZ (US); Ziyu ZHOU, Shanghai (CN); Jianming LIANG, Scottsdale, AZ (US)

(72) Inventors: Jiaxuan PANG, Tempe, AZ (US); DongAo MA, Tempe, AZ (US); Ziyu ZHOU, Shanghai (CN); Jianming LIANG, Scottsdale, AZ (US)

(73) Assignee: Arizona Board of Regents on behalf of Arizona State University, Scottsdale, AZ (US)

(21) Appl. No.: 19/207,215

(22) Filed: May 13, 2025

Related U.S. Application Data
(60) Provisional application No. 63/647,995, filed on May 15, 2024.

(51) Int. Cl. G06T 15/08; G06N 3/0895

(52) U.S. Cl. CPC G06T 15/08; G06N 3/0895

(57) ABSTRACT

A self-supervised learning framework learns fine-grained features within, high-level global features of, sub-volume spatial relationships in, and contextual relationship features of, anatomical structures in medical images of a plurality of patients. The framework receives a plurality of CT 3D volumes for the plurality of patients (“patient volumes”), learns sub-volume spatial relationships within the patient volumes through 3D sub-volume order prediction, learns fine-grained image features within the patient volumes through volume appearance recovery, learns high-level global image features, and delineating local features, of anatomical structures in the patient volumes by maximizing an agreement between two spatially related views through a student-teacher network of the self-supervised learning framework, and generates local features based on sub-volumes by aligning shared local views within two spatially related views.

### CLAIM OF PRIORITY

This application claims the benefit of U.S. Provisional Patent Application No. 63/647,995, filed May 15, 2024, entitled “SYSTEMS, METHODS, AND APPARATUSES FOR LEARNING ANATOMICAL CONSISTENCY, SUB-VOLUME SPATIAL RELATIONSHIPS AND FINE-GRAINED APPEARANCE FOR COMPUTED TOMOGRAPHY IMAGES”, the disclosure of which is incorporated by reference herein in its entirety.

### GOVERNMENT RIGHTS AND GOVERNMENT AGENCY SUPPORT NOTICE

This disclosure was made with government support under R01 HL128785 awarded by the National Institutes of Health. The government has certain rights in the disclosure.

### COPYRIGHT NOTICE

This document contains material which is subject to copyright protection. The copyright owner has no objection to the facsimile reproduction by anyone of the document as it appears in the Patent and Trademark Office records, but otherwise reserves all copyright rights whatsoever.

### TECHNICAL FIELD

Embodiments of the disclosure relate to systems, methods, and apparatuses for implementing a self-supervised learning framework to learn fine-grained features, high-level global features, sub-volume spatial relationship features, and contextual relationship features of anatomical structures in medical images of patients.

### BACKGROUND

Annotating medical images is a laborious, time-consuming task that demands specialized expertise. As a result, self-supervised learning has emerged as a transformative paradigm which enables the deep learning model to autonomously learn from data without expert labels.

### BRIEF DESCRIPTION OF THE DRAWINGS

Embodiments are illustrated by way of example, and not by way of limitation, and can be more fully understood with reference to the following detailed description when considered in connection with the figures in which:

FIG. 1 depicts CT volumes of three patients across three views.

FIG. 2 depicts acquisition of anatomical knowledge through two learning phases according to embodiments of the disclosure.

FIG. 3 depicts full fine tuning and linear probing evaluation on single-organ segmentation tasks according to embodiments of the disclosure.

FIG. 4 graphically depicts embodiments that surpass the State of the Art (SoTA) Swin UNETR in label efficient transfer learning on AMOS2022, underscoring the robust features learned from anatomical consistency, sub-volume spatial relationships, and fine-grained appearance.

FIG. 5 presents Table 1, which shows the disclosed embodiments excel in both fully and self-supervised techniques, achieving the highest average Dice score in segmenting all organs.

FIG. 6 presents Table 2, showing results of a study on different learning tasks and strategies using BTCV, wherein the average Dice performance in segmenting all organs is reported, with the best performance highlighted in bold.

FIG. 7 presents Table 3, showing a comparison between 1D and 3D volume coordinates with the best performances highlighted in bold.

FIG. 8 presents example pseudo-code for one round of ASA alternate pretraining according to the disclosed embodiments.

### DETAILED DESCRIPTION

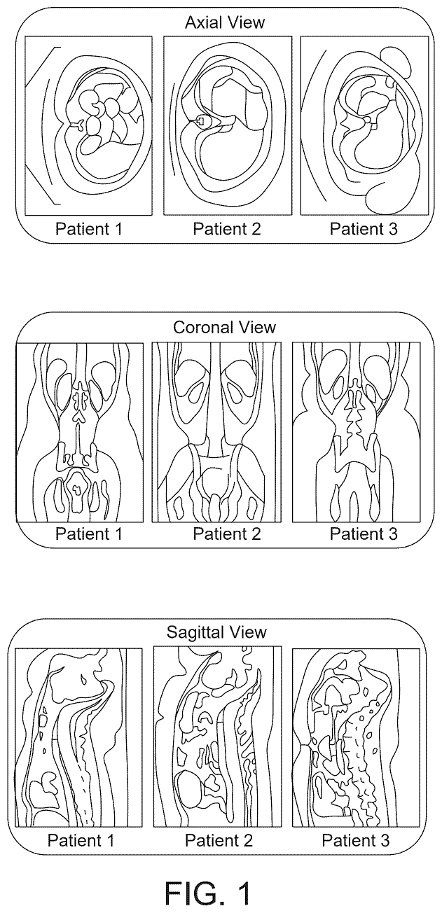

FIG. 1 depicts Computed Tomography (CT) volumes or images of three patients across three views: an axial view, a coronal view, and a sagittal view. Segmenting medical images requires not only understanding macroscopic anatomical patterns but also microscopic textural details. Given the intriguing symmetry and recurrent patterns inherent in medical images, the disclosed embodiments involve a model that encompasses not only global anatomical information but also sub-volume relationships and fine-grained appearance information. As depicted in FIG. 1, substantial similarities are evident in the axial, coronal, and sagittal views across diverse patients in CT images of anatomical structures. A robust model according to the disclosed embodiments grasp the overarching concept of shared appearances and features (i.e., anatomical structures) in CT volumes across all patients. Furthermore, leveraging symmetry and the recurring nature of body or anatomical structures, a novel model according to the disclosed embodiments effectively identifies high-level anatomical structures and intra-volume spatial relationships. However, despite the significant similarities observed across all CT volumes, there are still subtle differences present across each individual. Thus, the model possesses the capability to capture fine-grained features to discern and account for patient-level distinctions.

In organ segmentation, illustrated in FIG. 1, larger organs such as the liver and the spleen exhibit larger regions of interest, while smaller organs like the esophagus and adrenal glands have a smaller region of interest, necessitating more meticulous attention. An effective model captures organ-specific relationships and appearance features and delivers precise pixel-level details of small regions. Therefore, the disclosed embodiments provide a self-supervised learning framework that can acquire nuanced fine-grained features, comprehend high-level global features, emphasize local-level embeddings, and capture contextual relationship features.

To achieve superior performance, deep learning relies on copious, high-quality, annotated data, but manually annotating medical images is tedious, laborious, and time-consuming, demanding specialized expertise, especially for segmentation tasks. Segmenting medical images requires not only understanding macroscopic anatomical patterns but also microscopic textural details. Given the intriguing symmetry and recurrent patterns inherent in medical images, the disclosed embodiments provide a powerful deep model that exploits high-level context, spatial relationships in anatomy, and low-level, fine-grained, textural features in tissues in a self-supervised manner. This is accomplished using a novel self-supervised learning approach referred to herein as ASA to learn Anatomical consistency, Sub-volume spatial relationships, and fine-grained Appearance for three-dimensional (3D) computed tomography (CT) images, according to the disclosed embodiments. The novelty of ASA stems from its utilization of intrinsic properties of medical images, with a specific focus on computed tomography volumes. ASA enhances the model's learning capabilities, encompassing high-level global features, sub-volume relationships, and intricate appearance features. Extensive experimental results, described below, validate the robustness, effectiveness, and efficiency of the pretrained ASA model.

ASA learns anatomical consistency, sub-volume spatial relationships, and fine-grained appearance. ASA incorporates four learning perspectives:
(1) capturing sub-volume relationships through 3D sub-volume order prediction,
(2) depicting fine-grained features within volumes through volume appearance recovery,
(3) comprehending high-level global features by maximizing the agreement between two spatially related views using the student-teacher network, and
(4) acquiring local features at the sub-volume level by aligning the shared local views within two spatially related views.

Experimental results described below show that ASA outperforms both fully and self-supervised baselines on average in multi-organ segmentation tasks. The full finetuning and linear-probing evaluations on single-organ segmentation tasks demonstrate that ASA provides generalized representations. Furthermore, ASA demonstrates superior label efficiency in multi-organ segmentation challenges.

ASA is different from the distorted image recovery task by focusing on reconstructing the correct volume from a set of displaced sub-volumes to capture fine-grained volume appearances and underlying structures. ASA is also distinguished from contrastive learning methods which aim to maximize agreement between two positive views, by further aligning the shared local views within these views. Moreover, ASA diverges from image context learning by incorporating a student-teacher network to optimize global and local consistency between two spatially related views, thereby facilitating the acquisition of generalized volume features. Informed by Ma, D., Pang, J., Gotway, M. B., Liang, J., “Foundation Ark: Accruing and Reusing Knowledge for Superior and Robust Performance,” International Conference on Medical Image Computing and Computer-Assisted Intervention, pp. 651-662, Springer (2023), where the cyclic pre-training strategy and the student-teacher networks have been demonstrated effective in accumulating knowledge across various tasks, ASA and associated work offers an alternate learning strategy to enhance self-supervised learning from multiple perspectives, providing the following contributions:
1) a novel vision transformer-based self-supervised learning framework for 3D medical images that simultaneously captures high-level anatomical information, intra-volume relationships, and fine-grained appearance features;
2) introduction of an alternate pretraining strategy involving a student-teacher network to facilitate learning from multiple perspectives;
3) comprehensive experiments showcasing the transferability of ASA across diverse single-organ and multi-organ segmentation tasks, surpassing the performance of multiple fully supervised and self-supervised methods; and
4) an efficient pretrained model that encapsulates rich semantic information, enhancing its utilization efficiency.

### 1. METHOD

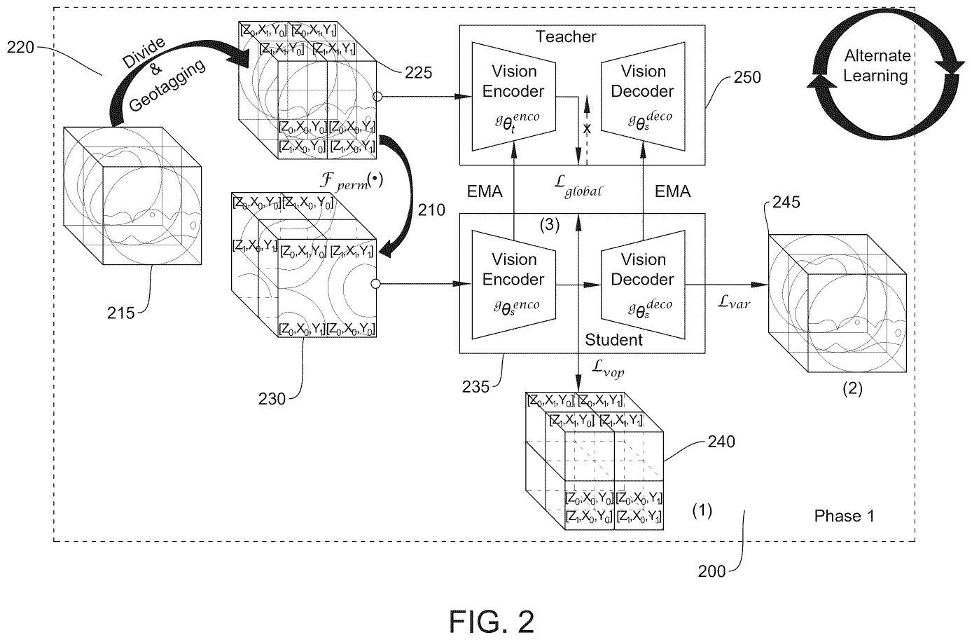

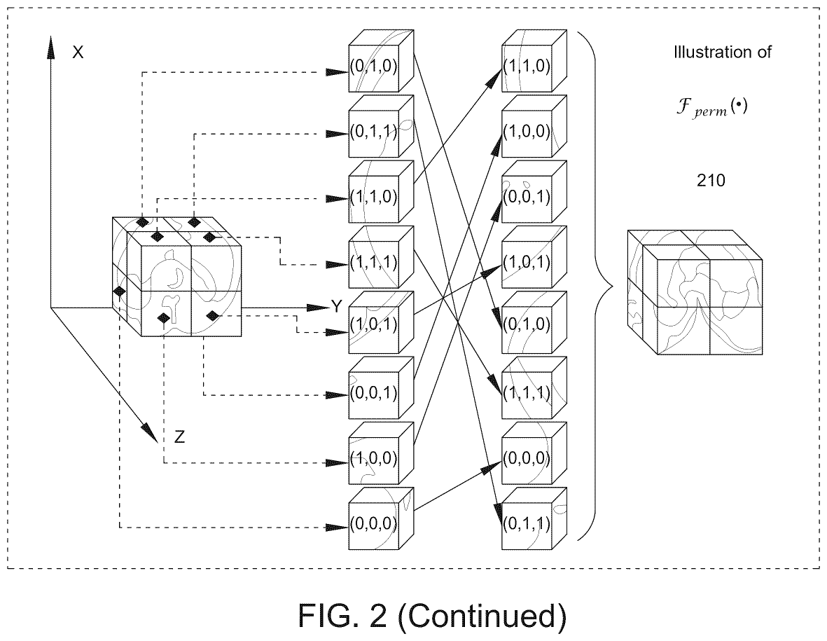

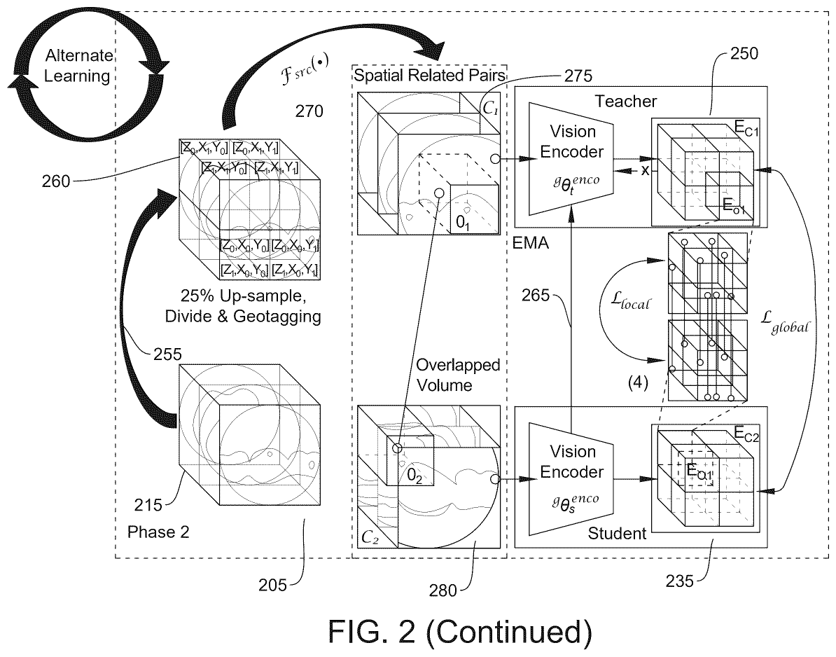

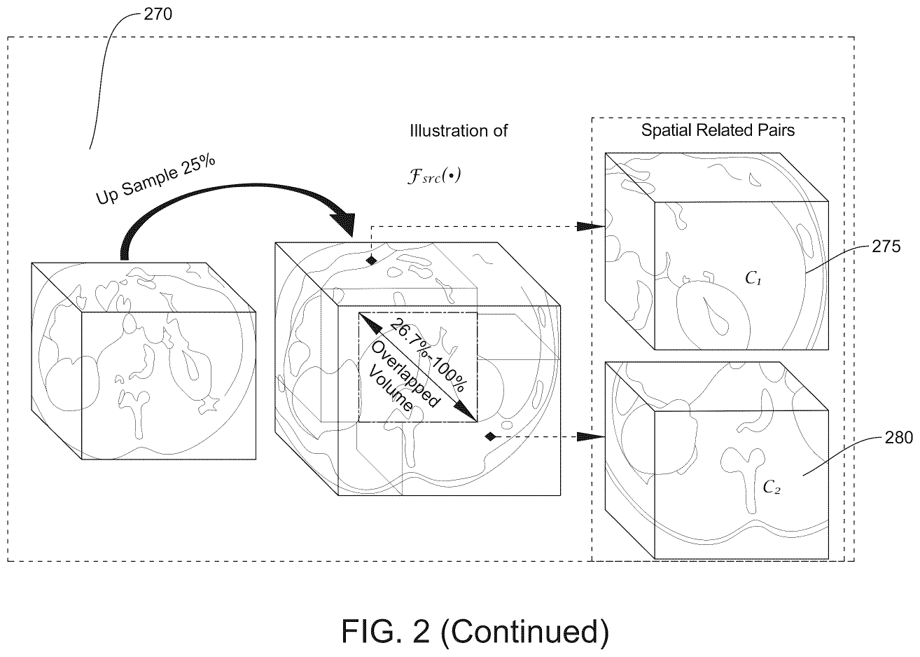

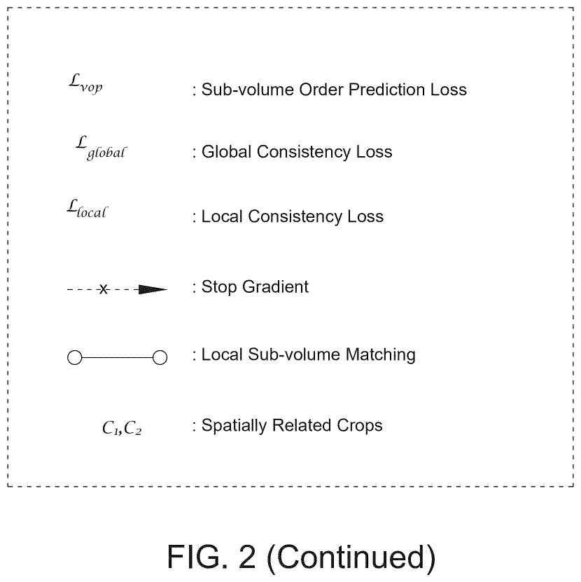

FIG. 2 graphically illustrates a disclosed embodiment of ASA which acquires anatomical knowledge through two learning phases. Phase 1 at 200 focuses on capturing sub-volume relationships via sub-volume order prediction, depicting fine-grained features via volume appearance recovery, and constructing high-level semantic context of an image via global feature alignment between the permuted and original volumes. Phase 2 at 205 enhances ASA's ability to summarize global features and delineate local features by optimizing the agreement between two spatially related views through the student-teacher learning paradigm.

During phase 1 at 200, a CT image 215 is divided at 220 into sub-volumes 225 whose order is distorted at 230 using Fₚₑᵣₘ (·) 210 before being fed into the student model 235. The model is then trained to predict at 240 the original sub-volume order 225 using the loss function Lᵥₒₚ (Sub-volume Order Prediction Loss), and to recover at 245 the volume's original appearance using the appearance recovery loss function Lᵥₐᵣ. Additionally, the sub-volumes 225 with the original order are fed at 250 into the teacher model to generate global features. These features are then utilized to compute the consistency loss L_{gCons} with the features obtained from the student model 235. In phase 2 at 205, the CT image is up-sampled and cropped at 255 into two spatially related (overlapped) views 260 using F_{src} (·), as illustrated at 270. Similar to step (3) described below, wherein the student network 235 aligns the original appearance embedding generated by the teacher 250 with the distorted appearance 230 embedding produced by the student network, these two views are fed into the student 235 and teacher 250 models to generate global features for maximizing agreement using L_{gCons}. Concurrently, the local features from the overlapped region of the two views are utilized to further enhance agreement between the student and teacher models, employing the loss L_{lCons}. In both phases 1 and 2, the teacher model 250 is updated after each iteration at 265 using exponential moving average (EMA) based on the student's weights. To stabilize and expedite training, the model alternates between these two learning phases 1 and 2. Once trained, the teacher model 250 is transferred to downstream tasks.

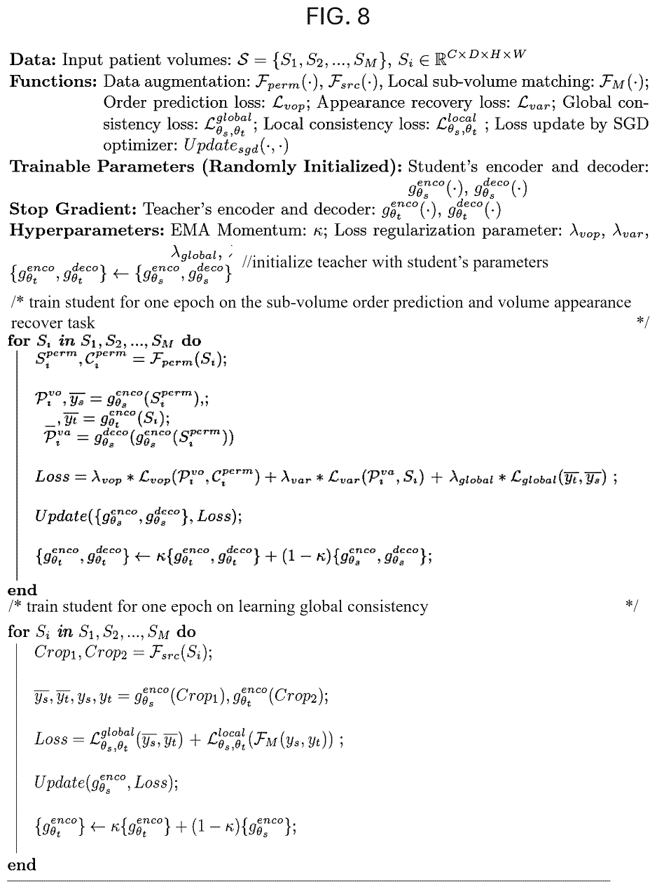

To develop a comprehensive understanding of anatomical structures depicted in CT images, particularly focusing on spatial relationships and fine-grained features, as demonstrated in phase 1 of FIG. 2, ASA incorporates the following components: (1) a sub-volume order prediction module 240 to capture intra-volume spatial relationships, and (2) a volume appearance recovery module 245 to represent volume-wise fine-grained features. Additionally, to establish a high-level semantic context within a CT image, ASA employs the student-teacher learning paradigm (with respective student 235 and teacher 250 modules), (3) aligning the global features extracted by the student's encoder with permuted volumes to those of the teacher's encoder with original volumes. Furthermore, to enhance ASA's ability to summarize global features and delineate local features, as depicted in phase 2 of FIG. 2, (4) spatially related cropping is employed, maximizing the agreement between teacher and student by aligning global and local features extracted from two spatially related crops C₁ and C₂ as respectively depicted at 275 and 280. The following description provides further details of ASA according to embodiments of the disclosure, in keeping with the example, detailed, training pseudo-code presented in FIG. 8 for one round of pretraining.

Learning intra-volume relationship and fine-grained appearance. In phase 1, depicted at 200 in FIG. 2, the original volume 215 is given to the teacher network 250, generating embedding of the original volume appearance, while the order-distorted volume 230, obtained by Fₚₑᵣₘ (·) 210, is fed to the student network 235. The objective of sub-volume order prediction module 240 is to anticipate the accurate 3D coordinates of a sub-volume from its appearance (phase 1, (1)), while volume appearance recovery module 245 endeavors to rebuild the original volume from an order-distorted one (phase 1, (2)). Meanwhile, to stabilize the reconstruction process and ensure maximum preservation of global features, embodiments instruct the student network 235 to align the original appearance embedding generated by the teacher with the distorted appearance embedding produced by the student network (phase 1, (3)).

Acquiring global and local embedding consistency from two related views. As depicted in phase 2 of FIG. 2, two spatially related crops C₁ 275 and C₂ 280, obtained at 270 by F_{src} (·), are input to the teacher 250 and student 235 networks, respectively. The objective of global embedding consistency is to enhance the general embedding level agreement between these two spatially related crops C₁ and C₂. To ensure alignment of the local embedding, embodiments employ a sub-volume matching process that maximizes agreement between the local embeddings, generated from two overlapped sub-volumes, showing near phase 2, (4).

Overall training scheme. As depicted in FIG. 2, embodiments conduct pretraining of the student network by alternately propagating the loss

$${L}_{vopar} = {λ}_{vop} * {L}_{vop} + {λ}_{var} * {L}_{var} + {λ}_{global} * {ℒ}_{{θ}_{\text{?}} , {θ}_{\text{?}}}^{global}$$

$$\text{?} \text{indicates text missing or illegible when filed}$$

in phase 1 and the loss

$${L}_{consistency} = {λ}_{global} * {ℒ}_{{θ}_{\text{?}} , {θ}_{\text{?}}}^{global} + {λ}_{local} * {ℒ}_{{θ}_{\text{?}} , {θ}_{\text{?}}}^{local}$$

$$\text{?} \text{indicates text missing or illegible when filed}$$

in phase 2, where λᵥₒₚ, λᵥₐᵣ, λ_{global} and λ_{local} are regularization factors contributing to the importance of the learning task. θₛ and θₜ are student and teacher networks, respectively. Embodiments optimize

$${ℒ}_{{θ}_{\text{?}} , {θ}_{\text{?}}}^{global}$$

$$\text{?} \text{indicates text missing or illegible when filed}$$

by minimizing l₂ distance between the predicted volume and the original volume, and both

$${ℒ}_{{θ}_{\text{?}} , {θ}_{\text{?}}}^{global} and {ℒ}_{{θ}_{\text{?}} , {θ}_{\text{?}}}^{local}$$

$$\text{?} \text{indicates text missing or illegible when filed}$$

are optimized by minimizing the l₂ distance between two normalized volume and sub-volume embeddings, respectively. Finally, embodiments define Lᵥₒₚ as a regression task by minimizing 12 distance between the predicted sub-volume coordinates and the randomly shuffled coordinates generated by Fₚₑᵣₘ (·). Only the student's encoder and decoder are updated by Lᵥₒₚₐᵣ, while L_{consistency} updates only the student's encoder. The weights of all learnable networks are shared between the two phases. Additionally, to summarize and consolidate the knowledge acquired from the two phases, embodiments employ a teacher model with the same architecture as the student model. The teacher network is updated using an exponential moving average (EMA) based on the learning experience of the student. Consequently, the learned sub-volume-wise relationships, volume-wise fine-grained features, and overall context are refined within the teacher model for future application-specific downstream tasks.

### 2. EXPERIMENTS AND RESULTS

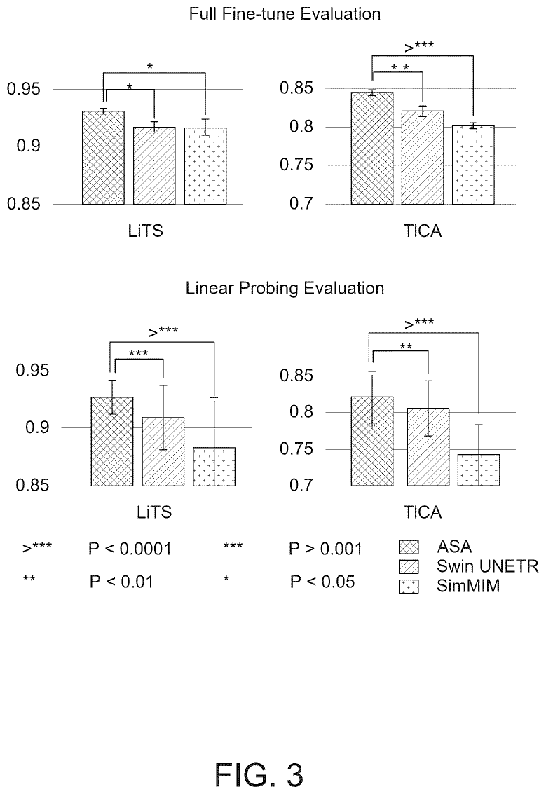

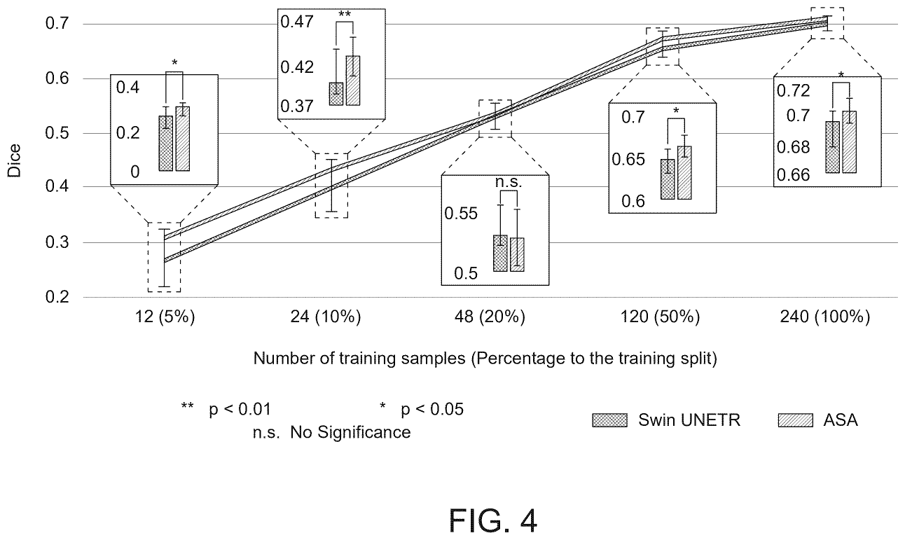

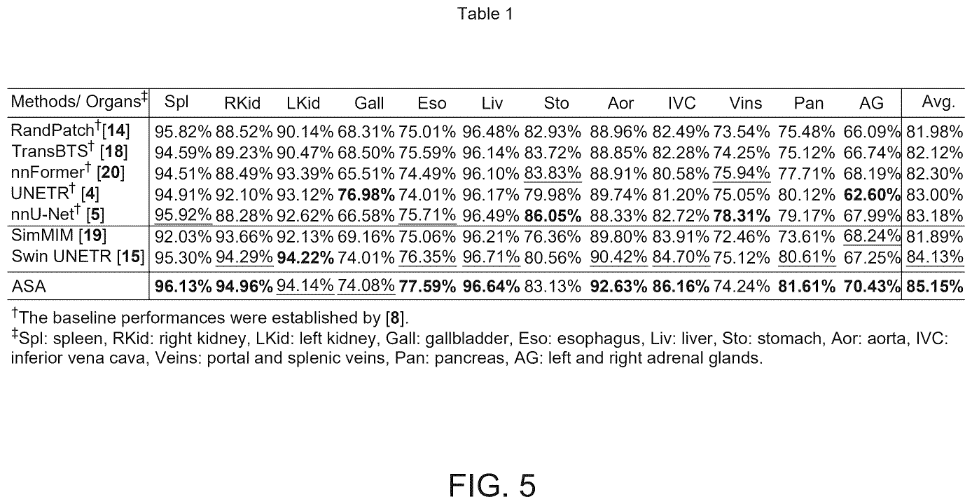

ASA underwent independent pretraining on AMOS2022 (Ji, Y., Bai, H., Yang, J., Ge, C., Zhu, Y., Zhang, R., Li, Z., Zhang, L., Ma, W., Wan, X., et al., “Amos: A Large-Scale Abdominal Multi-Organ Benchmark for Versatile Medical Image Segmentation,” arXiv preprint arXiv: 2206.08023 (2022)) and LUNA16 (Setio, A. A. A., Traverso, A., De Bel, T., Berens, M. S., Van Den Bogaard, C., Cerello, P., Chen, H., Dou, Q., Fantacci, M. E., Geurts, B., et al., “Validation, comparison, and combination of algorithms for automatic detection of pulmonary nodules in computed tomography images: the LUNA16 challenge,” Medical Image Analysis 42, 1-13 (2017)) datasets for different evaluation purposes. In phase 1, volumes were resized to 1×128×128×128, with the sub-volume size 16×16×16, leading to 512 unique shuffle-able sub-volumes and coordinates. The volume in phase 2 was up-sampled to 160×160×160 before two spatially related crops sized 1×128×128×128 were obtained. The Swin UNETR architecture (Hatamizadeh, A., Nath, V., Tang, Y., Yang, D., Roth, H. R., Xu, D., “Swin UNETR: Swin Transformers for Semantic Segmentation of Brain Tumors in MRI images,” In: International MICCAI Brainlesion Workshop. pp. 272-284. Springer (2021)) was employed as both the student and teacher networks. The model underwent a thorough comparison with both fully-supervised and self-supervised baselines, revealing its superior performance across various metrics related to multi-organ segmentation task (FIG. 5, Table 1), full fine tuning, and linear probing evaluation on single organ segmentation tasks (FIG. 3), as well as the label efficiency examination task (FIG. 4), in which embodiments surpassed the SoTA Swin UNETR in label efficient transfer learning on AMOS2022, underscoring its robust features learned from anatomical consistency, sub-volume spatial relationships, and fine-grained appearance. Detailed pre-training and evaluation protocols are discussed below.

In Table 1 presented in FIG. 5, ASA excels in both fully and self-supervised techniques, achieving the highest average Dice score in segmenting all organs. With comprehensive learning objectives encompassing anatomical consistency, sub-volume spatial relationships, and fine-grained appearance, ASA outperforms competitors in segmenting nine out of twelve organs fully supervised and ten out of twelve organs self-supervised.

### A. ASA Outperforms State-of-the-Art Fully/Self-Supervised Methods on Multi-Organ Segmentation Challenge on Average.

Experimental Setup: To showcase the performance enhancements achieved through ASA pretraining, the ASA model pretrained on the AMOS2022 was compared with state-of-the-art fully supervised and self-supervised models. Additionally, SimMIM baseline was trained on the same dataset, adhering to the official implementation and implementing the method in 3D on the Swin UNETR architecture. A 50% masking ratio was utilized, with each mask sized 16×16×16.

Swin UNETR underwent a pretraining phase involving three common self-supervised learning tasks on five publicly accessible CT datasets. The pretrained model was obtained from its official GitHub release. Given the availability of only encoder weights, the decoder part was initialized randomly for all subsequent evaluations in downstream tasks. For a fair comparison, the fully-supervised baseline performances were obtained and the training/validation/testing split proposed by Liu (Liu, J., Zhang, Y., Chen, J. N., Xiao, J., Lu, Y., A Landman, B., Yuan, Y., Yuille, A., Tang, Y., Zhou, Z., “CLIP-Driven Universal Model for Organ Segmentation and Tumor Detection,” In: Proceedings of the IEEE/CVF International Conference on Computer Vision. pp. 21152-21164 (2023)) was followed. All models were fully fine-tuned on the same training split and followed the same evaluation protocol.

Result and Analysis: As depicted in Table 1, the ASA model exceled beyond all reported methods on thirteen organ segmentation tasks (left and right adrenal glands combined) for the BTCV validation set. Particularly noteworthy is its superior performance over all five fully supervised learning methods in segmenting nine out of twelve organs. This underscores the effectiveness of the method according to the disclosed embodiments, which is pretrained on only one abdomen dataset, in acquiring more generic appearance features for a variety of abdomen organs. Additionally, the model according to the embodiments surpasses the self-supervised pretraining methods SimMIM and Swin UNETR, recognized as state-of-the-art (SoTA) in 2D natural/medical imaging and 3D medical imaging domains, respectively. The substantial margin by which the method disclosed herein outperforms SimMIM underscores the efficacy of learning anatomical relationships. Furthermore, it is noteworthy that the model outperforms Swin UNETR which was pretrained via three proxy tasks to learn volume-level discriminative and rotation-invariant features for the thoracic and abdominal regions using five datasets. This observation implies that more robust features can be learned by capturing anatomical structure via sub-volume order prediction and depicting fine-grained appearance features results via volume appearance recovery.

### B. ASA Offers Generalized Representations for Single-Organ Segmentation Tasks Under Both Fully Fine-Tuned and Linear Probing Scenarios.

Experimental Setup: ASA's generalizability was assessed by transferring the pre-trained AMOS2022 ASA model and two self-supervised baselines to pancreas and liver segmentation tasks. Using full finetuning and linear probing, the data split outlined in Liu was followed and evaluated on the Pancreas-CT dataset (80 CT scans) and LiTS dataset (130 CT scans). For linear probing, the pretrained model's weights were initialized and then the backbone frozen while allowing the decoder to undergo fine-tuning.

Result and Analysis: As depicted in FIG. 3, the ASA model surpassed both base-line self-supervised learning methods in both full finetuning and linear probing evaluation. As the liver is a sizable organ in the abdominal region, all models demonstrate high performances as measured by the Dice score. Notably, the method according to the disclosed embodiments attains the highest score, underscoring its superiority in delineating intricate edge details. In pancreas segmentation, the novel approach described herein outperformed SimMIM by a significant margin and surpassed Swin UNETR by a more modest margin, highlighting the enhanced adaptability of features acquired through the method, which effectively captures spatial relationships and fine-grained features.

### C. ASA Exhibits Better Label Efficiency on Multi-Organ Segmentation Challenge.

Experimental Setup: ASA and Swin UNETR pretrained models were fined tuned on subsets comprising 12 (5%), 24 (10%), 48 (20%), 120 (50%), and 240 (100%) randomly selected samples from the official training split of AMOS2022. To ensure fairness across diverse random samples, five independent runs were conducted and their average performances reported. The mean Dice scores of 15 organ segmentation performances in this task are reported.

Result and Analysis: ASA demonstrates its superiority by surpassing Swin UNETR, a state-of-the-art self-supervised learning method that comprises three learning objectives on 3D medical segmentation task benchmarks. This highlights the superiority of the ASA model, which offers richer information that can be utilized more effectively. As depicted in FIG. 4, in lower data regimes with 12 (5%) and 24 (10%) training samples, the ASA model significantly outperforms Swin UNETR. Although the performance is comparable when training with 48 (20%) samples, ASA still outperforms Swin UNETR with a further increase in training samples (50% and 100%). This emphasizes the effectiveness of the method in extracting fine-grained features and organ appearance information, even when pretrained on fewer datasets.

### D. Ablation Study: Comparison Among Different Learning Tasks.

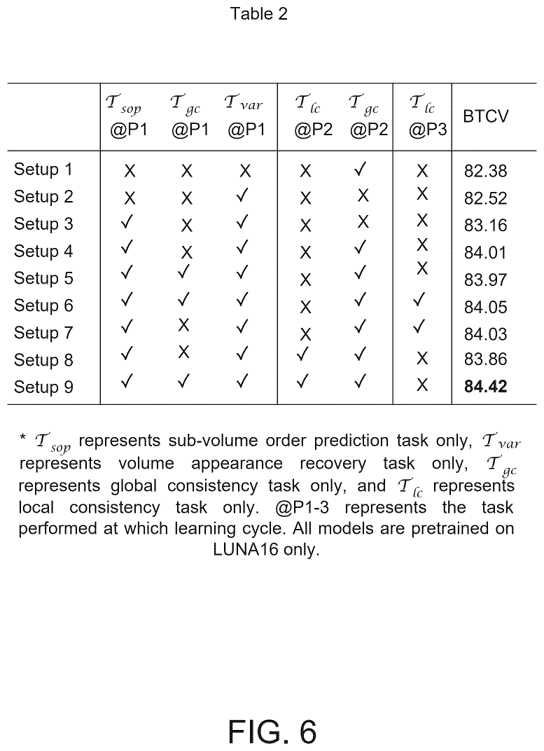

In anticipation of various combinations of training tasks and learning strategies, extensive ablation studies were conducted to showcase the superiority of the current ASA setup in the Beyond the Cranial Vault (BTCV) task. As illustrated in Table 2 presented in FIG. 6, the model trained solely with two crop consistency task (Setup 1) or focused solely on recovering volume appearance (Setup 2) shows the lowest performance. Introducing sub-volume order prediction alongside appearance recovery (Setup 3) leads to a slight enhancement in performance, underscoring the significance of learning sub-volume level relationships. The integration of cyclic training, coupled with the infusion of global consistency in both cycles (Setups 4 and 5), results in an additional performance boost. However, the inclusion of local consistency and the addition of a third learning cycle (Setups 6 and 7) do not yield any discernible benefits. Lastly, the current ASA setup (Setup 9), which incorporates Tₛₒₚ, Tᵥₐᵣ, and T_{gc} in the first learning cycle, and T_{gc} and T_{lc} in the second learning cycle, showcases the most prominent performance. Notably, there is a significant performance drop upon the removal of T_{gc} in the first cycle, highlighting the importance of consistency between the original view embedding from the teacher and the expected view embedding from the student network.

### E. Ablation Study: 1D Sub-Volume Sequences and 3D Sub-Volume Co-Ordinates Prediction.

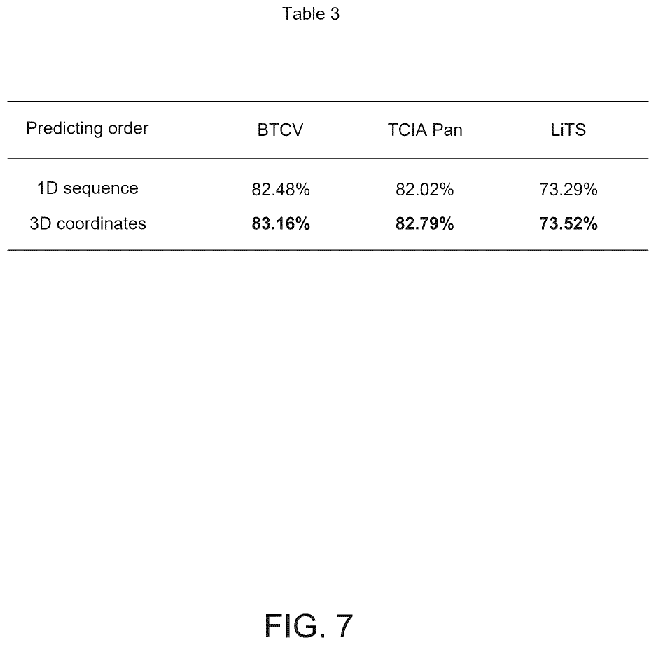

The efficacy of employing 1D sub-volume order presentation (e.g. 1,2,3, . . . , k), and 3D sub-volume order presentation (e.g. (0,0,0), . . . , (3,3,5), . . . , (z,x,y)) was studied. All models presented in Table 3 presented in FIG. 7 are pretrained on LUNA16 only with a combination of volume appearance recovery and sub-volume order prediction tasks. The results indicate that when making predictions based on 3D sub-volume order presentation, performance across all downstream tasks consistently surpasses that achieved with 1D sub-volume order prediction. This observation underscores the importance of utilizing 3D sub-volume order presentation for enhanced model performance.

### 3. CONCLUSION

Thus, a novel self-supervised learning method is described, referred to herein as ASA, capitalizing on the unique attributes of medical images to acquire robust global features, intra-volume relationships, and detailed appearance features. Furthermore, ASA introduces a novel pretraining paradigm, employing a student-teacher network to alternately attain diverse learning perspectives. Thoroughly examined through extensive experiments, ASA has proven its effectiveness and efficiency.

The disclosed embodiment includes a method performed by a system having at least a processor and a memory therein to execute instructions for a self-supervised learning framework to learn fine-grained features within, high-level global features of, sub-volume spatial relationships in, and contextual relationship features of, anatomical structures in medical images of a plurality of patients, comprising: receiving a plurality of computed tomography (CT) three-dimensional (3D) volumes for the plurality of patients (“patient volumes”); learning sub-volume spatial relationships within the patient volumes through 3D sub-volume order prediction; recovery; learning high-level global image features, and delineating local features, of anatomical structures in the patient volumes by maximizing an agreement between two spatially related views through a student-teacher network of the self-supervised learning framework; and generating local features based on sub-volumes by aligning shared local views within two spatially related views.

According to one embodiment, learning the sub-volume spatial relationships within the patient volumes and learning the fine-grained image features within the patient volumes are performed simultaneously using sub-volume order distortion.

According to one embodiment, learning high-level global image features of anatomical structures in the patient volumes by maximizing the agreement between two spatially related views through the student-teacher network of the self-supervised learning framework comprises learning high-level global image features of anatomical structures in the patient volumes by maximizing the agreement between features of two spatially related cropped views of the patient volumes.

According to one embodiment, learning sub-volume spatial relationships within the patient volumes through 3D sub-volume order prediction comprises, for each patient volume: dividing the patient volume into a plurality of sub-volumes whose order is distorted; training the learning framework to predict a correct order of sub-volumes for the patient volume using a sub-volume order prediction loss function; and recovering the patient volume using an appearance recovery loss function.

According to this embodiment, learning the high-level global image features may comprise learning high-level global image features based on the predicted correct orders of the sub-volumes for the patient volumes.

According to one embodiment, learning high-level global image features, and delineating local features, of anatomical structures in the patient volumes by maximizing the agreement between two spatially related views through the student-teacher network of the self-supervised learning framework comprises up-sampling and cropping the patient volume into the two spatially related views.

According to this embodiment, learning the high-level global image features of anatomical structures in the patient volumes by maximizing the agreement between two spatially related views through the student-teacher network of the self-supervised learning framework may comprise providing the two spatially related views into the student-teacher network to generate the high-level global image features that maximize the agreement between the two spatially related views.

According to this embodiment, learning the high-level global image features and delineating the local features of the anatomical structures may comprise cropping each of the two spatially related views into a first cropped and a second cropped spatially related views, and wherein maximizing the agreement between the two spatially related views comprises maximizing the agreement between the first and second cropped spatially related views.

The disclosed embodiments contemplate a machine or system within which embodiments may operate, be installed, integrated, or configured. According to one embodiment, the system includes at least a processor and a memory therein to execute instructions including implementing any application code to perform any one or more of the methodologies discussed herein. Such a system may communicatively interface with and cooperatively execute with the benefit of remote systems, such as a user device sending instructions and data, or a user device to receive output from the system.

A bus interfaces various components of the system amongst each other, with any other peripheral(s) of the system, and with external components such as external network elements, other machines, client devices, cloud computing services, etc. Communications may further include communicating with external devices via a network interface over a LAN, WAN, or the public Internet.

In alternative embodiments, the system may be connected (e.g., networked) to other machines in a Local Area Network (LAN), an intranet, an extranet, or the public Internet. The machine may operate in the capacity of a server or a client machine in a client-server network environment, as a peer machine in a peer-to-peer (or distributed) network environment, as a server or series of servers within an on-demand service environment. Certain embodiments of the machine may be in the form of a personal computer (PC), a tablet PC, a set-top box (STB), a Personal Digital Assistant (PDA), a cellular telephone, a web appliance, a server, a network router, switch or bridge, computing system, or any machine capable of executing a set of instructions (sequential or otherwise) that specify and mandate the specifically configured actions to be taken by that machine pursuant to stored instructions. Further, the term “machine” shall also be taken to include any collection of machines (e.g., computers) that individually or jointly execute a set (or multiple sets) of instructions to perform any one or more of the methodologies discussed herein.

An exemplary computer system includes a processor, a main memory (e.g., read-only memory (ROM), flash memory, dynamic random access memory (DRAM) such as synchronous DRAM (SDRAM) or Rambus DRAM (RDRAM), etc., static memory such as flash memory, static random access memory (SRAM), volatile but high-data rate RAM, etc.), and a secondary memory (e.g., a persistent storage device including hard disk drives and a persistent database and/or a multi-tenant database implementation), which communicate with each other via a bus. Main memory includes code that implements the three branches of the SSL framework described herein, namely, the localizability branch, the composability branch, and the decomposability branch.

The processor represents one or more specialized and specifically configured processing devices such as a microprocessor, central processing unit, or the like. More particularly, the processor may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor may also be one or more special-purpose processing devices such as an application-specific integrated circuit (ASIC), a field programmable gate array (FPGA), a digital signal processor (DSP), network processor, or the like. The processor is configured to execute processing logic for performing the operations and functionality discussed herein.

The system may further include a network interface card. The system also may include a user interface (such as a video display unit, a liquid crystal display, etc.), an alphanumeric input device (e.g., a keyboard), a cursor control device (e.g., a mouse), and a signal generation device (e.g., an integrated speaker). According to an embodiment of the system, the user interface communicably interfaces with a user client device remote from the system and communicatively interfaces with the system via a public Internet.

The system may further include peripheral devices (e.g., wireless or wired communication devices, memory devices, storage devices, audio processing devices, video processing devices, etc.).

A secondary memory may include a non-transitory machine-readable storage medium or a non-transitory computer readable storage medium or a non-transitory machine-accessible storage medium on which is stored one or more sets of instructions (e.g., software) embodying any one or more of the methodologies or functions described herein. The software may also reside, completely or at least partially, within the main memory and/or within the processor during execution thereof by the system, the main memory and the processor also constituting machine-readable storage media. The software may further be transmitted or received over a network via the network interface card.

In addition to various hardware components depicted in the figures and described herein, embodiments further include various operations which are described herein. The operations described in accordance with such embodiments may be performed by hardware components or may be embodied in machine-executable instructions, which may be used to cause a specialized and special-purpose processor having been programmed with the instructions to perform the operations described herein. Alternatively, the operations may be performed by a combination of hardware and software. In such a way, the embodiments of the disclosure provide a technical solution to a technical problem.

Embodiments also relate to an apparatus for performing the operations disclosed herein. This apparatus may be specially constructed for the required purposes, or it may be a special purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMS, EEPROMs, magnetic or optical cards, or any type of media suitable for storing electronic instructions, each coupled to a computer system bus.

While the algorithms and displays presented herein are not inherently related to any particular computer or other apparatus, they are specially configured and implemented via customized and specialized computing hardware which is specifically adapted to more effectively execute the novel algorithms and displays which are described in greater detail herein. Various customizable and special purpose systems may be utilized in conjunction with specially configured programs in accordance with the teachings herein, or it may prove convenient, in certain instances, to construct a more specialized apparatus to perform the required method steps. The required structure for a variety of these systems will appear as set forth in the description. In addition, embodiments are not described with reference to any particular programming language. It will be appreciated that a variety of programming languages may be used to implement the teachings of the embodiments as described herein.

Embodiments may be provided as a computer program product, or software, that may include a machine-readable medium having stored thereon instructions, which may be used to program a computer system (or other electronic devices) to perform a process according to the disclosed embodiments. A machine-readable medium includes any mechanism for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.), a machine (e.g., computer) readable transmission medium (electrical, optical, acoustical), etc.

Any of the disclosed embodiments may be used alone or together with one another in any combination. Although various embodiments may have been partially motivated by deficiencies with conventional techniques and approaches, some of which are described or alluded to within the specification, the embodiments need not necessarily address or solve any of these deficiencies, but rather, may address only some of the deficiencies, address none of the deficiencies, or be directed toward different deficiencies and problems which are not directly discussed.

While the subject matter disclosed herein has been described by way of example and in terms of specific embodiments, it is understood that the claimed embodiments are not limited to the explicitly enumerated embodiments. To the contrary, the disclosure is intended to cover various modifications and similar arrangements as are apparent to those skilled in the art. Therefore, the scope of the appended claims is to be accorded the broadest interpretation to encompass all such modifications and similar arrangements. It is to be understood that the above description is intended to be illustrative, and not restrictive. Many other embodiments will be apparent to those of skill in the art upon reading and understanding the above description. The scope of the disclosed subject matter is therefore to be determined in reference to the appended claims, along with the full scope of equivalents to which such claims are entitled.

What is claimed is:

1. A method performed by a system having at least a processor and a memory therein to execute instructions for a self-supervised learning framework to learn fine-grained features within, high-level global features of, sub-volume spatial relationships in, and contextual relationship features of, anatomical structures in medical images of a plurality of patients, comprising:
receiving a plurality of computed tomography (CT) three-dimensional (3D) volumes for the plurality of patients (“patient volumes”);
learning sub-volume spatial relationships within the patient volumes through 3D sub-volume order prediction;
learning fine-grained image features within the patient volumes through volume appearance recovery;
learning high-level global image features, and delineating local features, of anatomical structures in the patient volumes by maximizing an agreement between two spatially related views through a student-teacher network of the self-supervised learning framework; and
generating local features based on sub-volumes by aligning shared local views within two spatially related views.

2. The method of claim 1, wherein learning the sub-volume spatial relationships within the patient volumes and learning the fine-grained image features within the patient volumes are performed simultaneously using sub-volume order distortion.

3. The method of claim 1, wherein learning high-level global image features of anatomical structures in the patient volumes by maximizing the agreement between two spatially related views through the student-teacher network of the self-supervised learning framework comprises learning high-level global image features of anatomical structures in the patient volumes by maximizing the agreement between features of two spatially related cropped views of the patient volumes.

4. The method of claim 1, wherein learning sub-volume spatial relationships within the patient volumes through 3D sub-volume order prediction comprises, for each patient volume:
dividing the patient volume into a plurality of sub-volumes whose order is distorted;
training the learning framework to predict a correct order of sub-volumes for the patient volume using a sub-volume order prediction loss function; and
recovering the patient volume using an appearance recovery loss function.

5. The method of claim 4 wherein learning the high-level global image features comprises learning high-level global image features based on the predicted correct orders of the sub-volumes for the patient volumes.

6. The method of claim 1 wherein learning high-level global image features, and delineating local features, of anatomical structures in the patient volumes by maximizing the agreement between two spatially related views through the student-teacher network of the self-supervised learning framework comprises up-sampling and cropping the patient volume into the two spatially related views.

7. The method of claim 6, wherein learning the high-level global image features of anatomical structures in the patient volumes by maximizing the agreement between two spatially related views through the student-teacher network of the self-supervised learning framework comprises providing the two spatially related views into the student-teacher network to generate the high-level global image features that maximize the agreement between the two spatially related views.

8. The method of claim 7, wherein learning the high-level global image features and delineating the local features of the anatomical structures comprises cropping each of the two spatially related views into a first cropped and a second cropped spatially related views, and wherein maximizing the agreement between the two spatially related views comprises maximizing the agreement between the first and second cropped spatially related views.

9. A system comprising:
a memory to store instructions;
a processor to execute the instructions stored in the memory;
a receive interface to receive computed tomography (CT) three-dimensional (3D) volumes for a plurality of patients (“patient volumes”);
wherein the system is configured for a self-supervised learning framework to learn fine-grained features within, high-level global features of, sub-volume spatial relationships in, and contextual relationship features of, anatomical structures in medical images of a plurality of patients, by executing the instructions via the processor for:
learning sub-volume spatial relationships within the patient volumes through 3D sub-volume order prediction;
learning fine-grained image features within the patient volumes through volume appearance recovery;
learning high-level global image features, and delineating local features, of anatomical structures in the patient volumes by maximizing an agreement between two spatially related views through a student-teacher network of the self-supervised learning framework; and
generating local features based on sub-volumes by aligning shared local views within two spatially related views.

10. The system of claim 9, wherein learning the sub-volume spatial relationships within the patient volumes and learning the fine-grained image features within the patient volumes are performed simultaneously using sub-volume order distortion.

11. The system of claim 9, wherein learning high-level global image features of anatomical structures in the patient volumes by maximizing the agreement between two spatially related views through the student-teacher network of the self-supervised learning framework comprises learning high-level global image features of anatomical structures in the patient volumes by maximizing the agreement between features of two spatially related cropped views of the patient volumes.

12. The system of claim 9, wherein learning sub-volume spatial relationships within the patient volumes through 3D sub-volume order prediction comprises, for each patient volume:
dividing the CT volume into a plurality of sub-volumes whose order is distorted;
training the learning framework to predict a correct order of sub-volumes for the patient volume using a sub-volume order prediction loss function; and
recovering the patient volume using an appearance recovery loss function.

13. The system of claim 12 wherein learning the high-level global image features comprises learning high-level global image features based on the predicted correct orders of the sub-volumes for the patient volumes.

14. The system of claim 9 wherein learning high-level global image features, and delineating local features, of anatomical structures in the patient volumes by maximizing an agreement between two spatially related views through a student-teacher network of the self-supervised learning framework comprises up-sampling and cropping the patient volume into the two spatially related views.

15. The system of claim 14, wherein learning the high-level global image features of anatomical structures in the patient volumes by maximizing the agreement between two spatially related views through the student-teacher network of the self-supervised learning framework comprises providing the two spatially related views into the student-teacher network to generate the high-level global image features that maximize the agreement between the two spatially related views.

16. The system of claim 15, wherein learning the high-level global image features and delineating the local features of the anatomical structures comprises cropping each of the two spatially related views into a first cropped and a second cropped spatially related views, and wherein maximizing the agreement between the two spatially related views comprises maximizing the agreement between the first and second cropped spatially related views.

17. A non-transitory computer-readable storage media having instructions stored thereupon that, when executed by a system having at least a processor and a memory therein, perform a self-supervised learning framework to learn fine-grained features within, high-level global features of, sub-volume spatial relationships in, and contextual relationship features of, anatomical structures in medical images of a plurality of patients, by executing the instructions via the processor comprising:
receiving computed tomography (CT) three-dimensional (3D) volumes for a plurality of patients (“patient volumes”);
learning sub-volume spatial relationships within the patient volumes through 3D sub-volume order prediction;
learning fine-grained image features within the patient volumes through volume appearance recovery;
learning high-level global image features, and delineating local features, of anatomical structures in the patient volumes by maximizing an agreement between two spatially related views through a student-teacher network of the self-supervised learning framework; and
generating local features based on sub-volumes by aligning shared local views within two spatially related views.

18. The non-transitory computer-readable storage media of claim 17, wherein learning the sub-volume spatial relationships within the patient volumes and learning the fine-grained image features within the patient volumes are performed simultaneously using sub-volume order distortion.

19. The non-transitory computer-readable storage media of claim 17, wherein learning high-level global image features of anatomical structures in the patient volumes by maximizing the agreement between two spatially related views through the student-teacher network of the self-supervised learning framework comprises learning high-level global image features of anatomical structures in the patient volumes by maximizing the agreement between features of two spatially related cropped views of the patient volumes.

20. The non-transitory computer-readable storage media of claim 17, wherein learning sub-volume spatial relationships within the patient volumes through 3D sub-volume order prediction comprises, for each patient volume:
dividing the patient volume into a plurality of sub-volumes whose order is distorted;
training the learning framework to predict a correct order of sub-volumes for the patient volume using a sub-volume order prediction loss function; and
recovering the patient volume using an appearance recovery loss function.
